(19) United States
(12) Patent Application Publication
(10) Pub. No.: US 2023/0236166 A1
(43) Pub. Date: Jul. 27, 2023

(54) NON-INVASIVE ANALYTE SENSOR WITH MULTIPLE SENSOR ASSEMBLIES

(71) Applicant: Know Labs, Inc., Seattle, WA (US)

(72) Inventor: Phillip BOSUA, Seattle, WA (US)

(21) Appl. No.: 17/584,870

(22) Filed: Jan. 26, 2022

(51) Int. Cl. G01N 33/487

(52) U.S. Cl. CPC G01N 33/48792; G01N 33/48778; G01N 33/49

(57) ABSTRACT

A non-invasive analyte sensor that includes first and second non-invasive analyte sensor assemblies each of which can emit transmit signals that are in a radio or microwave frequency range of the electromagnetic spectrum into a target and can detect responses resulting from emission of the transmit signals into the target.

### FIELD

This technical disclosure relates to apparatus, systems and methods of detecting an analyte via spectroscopic techniques using non-optical frequencies such as in the radio or microwave frequency bands of the electromagnetic spectrum. More specifically, this disclosure relates to a analyte sensor that includes first and second non-invasive analyte sensor assemblies.

### BACKGROUND

A sensor that uses radio or microwave frequency bands of the electromagnetic spectrum for non-invasive collection of analyte data of a subject is disclosed in U.S. Pat. No. 10,548,503. Additional examples of sensors that purport to be able to use radio or microwave frequency bands of the electromagnetic spectrum to detect an analyte in a person are disclosed in U.S. Patent Application Publication 2019/0008422 and U.S. Patent Application Publication 2020/0187791.

### SUMMARY

This disclosure relates generally to apparatus, systems and methods of non-invasively detecting an analyte via spectroscopic techniques using non-optical frequencies such as in the radio or microwave frequency bands of the electromagnetic spectrum. A non-invasive analyte sensor described herein includes first and second non-invasive analyte sensor assemblies each of which can emit transmit signals that are in a radio or microwave frequency range of the electromagnetic spectrum into a target and can detect responses resulting from emission of the transmit signals into the target.

The use of first and second non-invasive analyte sensor assemblies provides a number of benefits. For example, one of the sensor assemblies can be used to detect a first analyte in a target and the other sensor assembly can be used to detect a second analyte in the target. One of the sensor assemblies can be configured to operate on a first part of the target, such as detecting an analyte in blood, while the other sensor assembly can be configured to operate on a second part of the target, for example interstitial fluid of the target to detect the same or different analyte as the first sensor assembly. One sensor assembly can be configured to detect an analyte from one depth of the target, such as the human body, while the other sensor assembly is configured to detect an analyte from a different depth of the human body. The sensor assemblies can be arranged to operate over different areas of the body and the readings from the sensor assemblies can be processed to generate a more accurate depiction, such as an average, of an analyte in the target. In addition, one sensor assembly can act as back-up for the other sensor assembly or be used to validate the reading of the other sensor assembly.

In one embodiment, a non-invasive analyte sensor can include first and second non-invasive analyte sensor assemblies, where each of the first and second non-invasive analyte sensor assemblies is configured to emit transmit signals that are in a radio or microwave frequency range of the electromagnetic spectrum into a target and configured to detect responses resulting from emission of the transmit signals into the target. In one embodiment, each of the first and second non-invasive analyte sensor assemblies can be disposed at a same side of the target. However, the first and second non-invasive analyte sensor assemblies can be disposed at other locations relative to the target.

In another embodiment, a non-invasive analyte sensor can include a first non-invasive analyte sensor assembly that is configured to emit first transmit signals that are in a radio or microwave frequency range of the electromagnetic spectrum into a first portion of a target and configured to detect first responses resulting from emission of the first transmit signals into the first portion of the target, and a second non-invasive analyte sensor assembly that is configured to emit second transmit signals that are in a radio or microwave frequency range of the electromagnetic spectrum into a second portion of the target and configured to detect second responses resulting from emission of the second transmit signals into the second portion of the target, the second portion of the target is spaced from the first portion of the target.

In an embodiment, the first non-invasive analyte sensor assembly and the second non-invasive analyte sensor assembly can be controlled to alternate with one another in emitting the transmit signals into the target while implementing a frequency sweep over a range of frequencies. In this embodiment, the first non-invasive analyte sensor assembly and the second non-invasive analyte sensor assembly would not simultaneously emit the transmit signals. In another embodiment, the first non-invasive analyte sensor assembly and the second non-invasive analyte sensor assembly can be controlled to simultaneously emit the transmit signals.

### DRAWINGS

FIG. 1 illustrates a first example of a non-invasive analyte sensor described herein.

FIG. 2 illustrates another example of a non-invasive analyte sensor described herein.

FIG. 3 illustrates another example of a non-invasive analyte sensor described herein.

FIG. 4 illustrates another example of a non-invasive analyte sensor described herein.

### DETAILED DESCRIPTION

The following is a detailed description of apparatus, systems and methods of detecting an analyte via spectroscopic techniques using non-optical frequencies such as in the radio or microwave frequency bands of the electromagnetic spectrum. An analyte sensor described herein includes at least first and second non-invasive analyte sensor assemblies, where each of the first and second non-invasive analyte sensor assemblies is configured to emit transmit signals that are in a radio or microwave frequency range of the electromagnetic spectrum into a target and configured to detect responses resulting from emission of the transmit signals into the target. In an embodiment, the analyte sensor can include more than two of the sensor assemblies.

Each one of the first and second non-invasive analyte sensor assemblies can include two or more antennas (also referred to as detector elements), at least one of which operates as a transmit antenna (or transmit element) and at least one of which operates as a receive antenna (or receive element). In another embodiment, each one of the first and second non-invasive analyte sensor assemblies can include a single antenna or detector element that is used for both transmitting and receiving signals. Or one of the first and second non-invasive analyte sensor assemblies can include two or more antennas, and one of the sensor assemblies can include a single antenna.

The analyte sensor is configured to transmit generated transmit signals that are in a radio or microwave frequency range of the electromagnetic spectrum from the antenna(s) into a target containing an analyte, and to also detect responses that result from transmission of the transmit signals into the target. Examples of detecting analytes using non-invasive spectroscopy sensors operating in the radio or microwave frequency range of the electromagnetic spectrum are described in U.S. Pat. Nos. 11,063,373, 11,058,331, 10,548,503, U.S. 2021/0259571, and U.S. Pat. No. 11,033,208, the entire contents of each are incorporated herein by reference.

The use of at least two non-invasive analyte sensor assemblies provides a number of benefits. For example, one of the sensor assemblies can be used to detect a first analyte, such as glucose, in a target and the other sensor assembly can be used to detect a second analyte, such as alcohol, white blood cells or luteinizing hormone in the target. Alternatively, one of the sensor assemblies can be configured to operate on a first part of the target, such as detecting an analyte in blood, while the other sensor assembly can be configured to operate on a second part of the target, for example detecting the same or different analyte in interstitial fluid of the target. Alternatively, one sensor assembly can be configured to detect an analyte from one depth of the target, such as the human body, while the other sensor assembly is configured to detect an analyte from a different depth of the human body. Alternatively, the sensor assemblies can be arranged to operate over different areas of the body and the readings from the sensor assemblies can be processed to generate a more accurate depiction of an analyte, such as glucose, in the target. For example, an average of the analyte can be determined. In addition, one sensor assembly can act as back-up for the other sensor assembly or be used to validate the reading of the other sensor assembly.

In one embodiment, the sensors described herein can be used to detect the presence of at least one analyte in a target. In another embodiment, the sensors described herein can detect an amount or a concentration of the at least one analyte in the target. The target can be any target containing at least one analyte that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, a fluid, for example blood, interstitial fluid, cerebral spinal fluid, lymph fluid or urine, human tissue, animal tissue, plant tissue, an inanimate object, soil, genetic material, or a microbe.

The detection by the sensors described herein can be non-invasive meaning that the sensor remains outside the target, such as the human body, and the detection of the analyte occurs without requiring removal of fluid or other removal from the target, such as the human body. In the case of sensing an analyte in the human body, this non-invasive sensing may also be referred to as in vivo sensing. In other embodiments, the sensors described herein may be used to sense an analyte in material that has been removed, for example from a human body.

The sensor assemblies can be located near the target and operated as further described herein to assist in detecting at least one analyte in the target. The sensor assemblies transmit signals, each of which may have at least two frequencies in the radio or microwave frequency range, toward and into the target. The signals with the at least two frequencies can be formed by separate signal portions, each having a discrete frequency, that are transmitted separately at separate times at each frequency. In another embodiment, the signals with the at least two frequencies may be part of complex signals that include a plurality of frequencies including the at least two frequencies. The complex signals can be generated by blending or multiplexing multiple signals together followed by transmitting the complex signals whereby the plurality of frequencies are transmitted at the same time. One possible technique for generating the complex signals includes, but is not limited to, using an inverse Fourier transformation technique. The sensor assemblies also detect responses resulting from transmission of the signals by the sensor assemblies into the target containing the at least one analyte of interest.

The signals detected by the sensor assemblies can be analyzed to detect the analyte(s) based on the intensities of the received signals and reductions in intensity at one or more frequencies where the analyte(s) absorbs the transmitted signals. An example of detecting an analyte using a non-invasive spectroscopy sensor operating in the radio or microwave frequency range of the electromagnetic spectrum is described in U.S. Pat. No. 10,548,503, the entire contents of which are incorporated herein by reference. The signals detected by the sensor assemblies can be complex signals including a plurality of signal components, each signal component being at a different frequency. In an embodiment, the detected complex signals can be decomposed into the signal components at each of the different frequencies, for example through a Fourier transformation. In an embodiment, the complex signals detected by the sensor assemblies can be analyzed as a whole (i.e. without demultiplexing the complex signal) to detect the analyte as long as the detected signals provide enough information to make the analyte detection. In addition, the signals detected by the sensor assemblies can be separate signal portions, each having a discrete frequency.

The analyte(s) can be any analyte(s) that one may wish to detect. The analyte(s) can be human or non-human, animal or non-animal, biological or non-biological. For example, the analyte(s) can include, but is not limited to, one or more of glucose, alcohol, white blood cells, or luteinizing hormone. The analyte(s) can include, but is not limited to, a chemical, a combination of chemicals, a virus, bacteria, or the like. The analyte(s) can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-βhydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

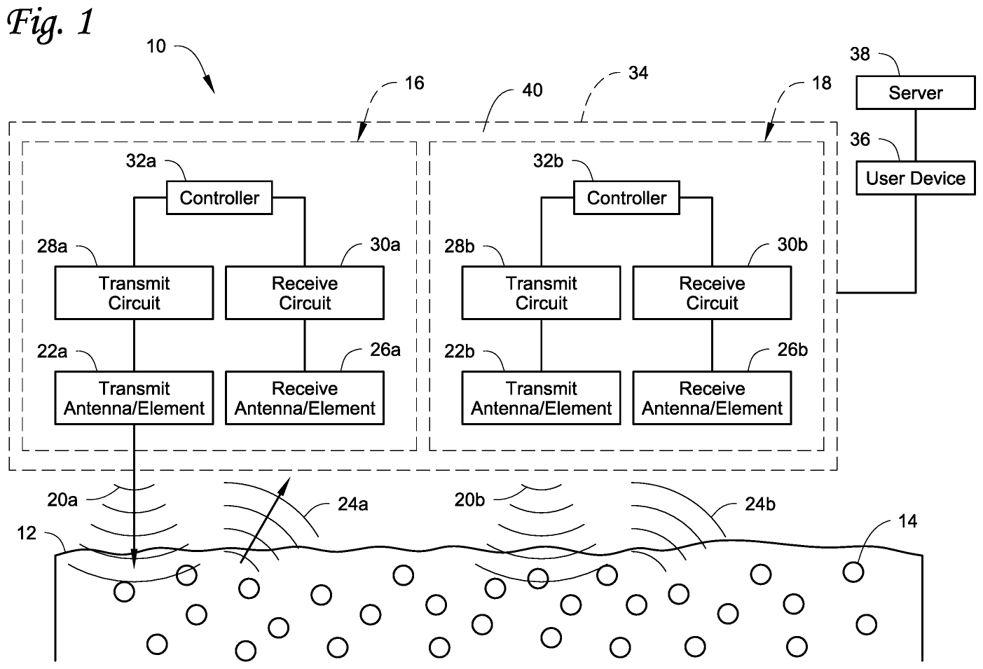

Referring now to FIG. 1, an embodiment of a non-invasive analyte sensor system with a non-invasive analyte sensor 10 is illustrated. The sensor 10 is depicted relative to a target 12 that contains an analyte of interest 14, for example an analyte in interstitial fluid in a human body. In this example, the sensor 10 is depicted as including first and second non-invasive analyte sensor assemblies 16, 18, respectively, that are disposed at the same side of the target 12 and are external to the target 12. Each of the first and second non-invasive analyte sensor assemblies 16, 18 is configured to emit transmit signals that are in a radio or microwave frequency range of the electromagnetic spectrum into a target and configured to detect responses resulting from emission of the transmit signals into the target.

Each one of the sensor assemblies 16, 18 illustrated in FIG. 1 operates by transmitting electromagnetic signals 20a, 20b in the radio or microwave frequency range of the electromagnetic spectrum toward and into the target 12 using at least one transmit element such as at least one transmit antenna 22a, 22b associated with the sensor assemblies 16, 18. Returning signal(s) 24a, 24b that result from the transmission of the transmitted signals 20a, 20b are detected by at least one receive element such as at least one receive antenna 26a, 26b associated with the sensor assembly 16, 18. The signals 24a, 24b detected by the receive antennas 26a, 26b can then be analyzed to detect one or more analytes.

Each of the sensor assemblies 16, 18 includes two or more antennas including at least one of which acts as the transmit antenna 22a, 22b and at least one of which acts as the receive antenna 26. The transmit antenna(s) 22a, 22b and the receive antenna(s) 26a, 26b can be located near the target 12 and operated as further described herein to assist in detecting at least one analyte in the target 12. The signals 20a, 20b transmitted by the transmit antennas 22a, 22b may have at least two frequencies in the radio or microwave frequency range. The signals 20a, 20b with the at least two frequencies can be formed by separate signal portions, each having a discrete frequency, that are transmitted separately at separate times at each frequency. In another embodiment, the signals with the at least two frequencies may be part of a complex signal that includes a plurality of frequencies including the at least two frequencies. The complex signal can be generated by blending or multiplexing multiple signals together followed by transmitting the complex signal whereby the plurality of frequencies are transmitted at the same time. One possible technique for generating the complex signal includes, but is not limited to, using an inverse Fourier transformation technique. The receive antennas 26a, 26b detect the returning signals 24a, 24b resulting from transmission of the signals 20a, 20b by the transmit antennas 22a, 22b into the target 12.

In each of the sensor assemblies 16, 18, the transmit antenna(s) 22a, 22b and the receive antenna(s) 26a, 26b are decoupled (which may also be referred to as detuned or the like) from one another. Decoupling refers to intentionally fabricating the configuration and/or arrangement of the transmit antenna(s) 22a, 22b and the receive antenna(s) 26a, 26b to minimize direct communication between the transmit antenna(s) 22a, 22b and the receive antenna(s) 26a, 26b, preferably absent shielding. Shielding between the transmit antenna(s) 22a, 22b and the receive antenna(s) 26a, 26b can be utilized. However, the transmit antenna(s) 22a, 22b and the receive antenna(s) 26a, 26b are decoupled even without the presence of shielding.

The signals 24a, 24b detected by the receive antennas 26a, 26b can be complex signals including a plurality of signal components, each signal component being at a different frequency. In an embodiment, the detected complex signals can be decomposed into the signal components at each of the different frequencies, for example through a Fourier transformation. In an embodiment, the complex signals detected by the receive antennas 26a, 26b can be analyzed as a whole (i.e. without demultiplexing the complex signal) to detect the analyte(s) as long as the detected signals 24a, 24b provide enough information to make the analyte detection. In addition, the signals 24a, 24b detected by the receive antennas 26a, 26b can be separate signal portions, each having a discrete frequency.

In an embodiment, the transmit antennas 22a, 22b can have the same configuration as each other, or the transmit antennas 22a, 22b can have different configurations from one another. In another embodiment, the receive antennas 26a, 26b can have the same configuration as each other, or the receive antennas 26a, 26b can have different configurations from one another. In another embodiment, the transmit antennas 22a, 22b can have the same configurations as the receive antennas 26a, 26b, or the transmit antennas 22a, 22b can have different configurations from the receive antennas 26a, 26b.

In an embodiment, the transmit signals 20a, 20b may be transmitted simultaneously (or substantially simultaneously) with the signals 24a, 24b being received simultaneously (or substantially simultaneously). For example, the antennas 22a, 22b can transmit the signals 20a, 20b at the same time or substantially the same time, with the return signals 24a, 24b also being detected by the antennas 26a, 26b at the same time or substantially at the same time. Alternatively, the transmit signals 20a, 20b can be sent sequentially or at separate times, which means that the return signals 24a, 24b would be detected by the antennas 26a, 26b sequentially or at different times. For example, in one embodiment, the antenna 22a can transmit the signal 20a first, followed thereafter by the antenna 22b transmitting the signal 20b, and the antenna 26a receiving the signal 24a and the antennas 26b thereafter receiving the signal 24b.

Returning to FIG. 1, each one of the sensor assemblies 16, 18 further includes a transmit circuit 28a, 28b, a receive circuit 30a, 30b, and a controller 32a, 32b. The analyte sensor can also include a power supply, such as a rechargeable battery (not shown in FIG. 1) that powers each sensor assembly 16, 18, or each sensor assembly 16, 18 can include its own battery. In some embodiments, power can be provided from mains power, for example by plugging the analyte sensor into a wall socket via a cord connected to the sensor. Each one of the sensor assemblies 16, 18 may be disposed side-by-side in a common housing 34 as depicted in dashed lines in FIG. 1. Alternatively, the sensor assemblies 16, 18 may be separate from each other in their own housings that are not physically connected to one another.

The transmit antennas 22a, 22b are positioned, arranged and configured to transmit the transmit signals 20a, 20b that are in the radio frequency (RF) or microwave range of the electromagnetic spectrum into the target 12. Each one of the transmit antennas 22a, 22b can be an electrode or any other suitable transmitter of electromagnetic signals in the radio frequency (RF) or microwave range. The transmit antennas 22a, 22b can have any arrangement and orientation relative to the target 12 that is sufficient to allow the analyte sensing to take place. In one non-limiting embodiment, the transmit antennas 22a, 22b can be arranged to face in a direction that is substantially toward the target 12.

The signals 20a, 20b transmitted by the transmit antennas 22a, 22b are generated by the transmit circuits 28a, 28b which are electrically connectable to the respective transmit antennas 22a, 22b. The transmit circuits 28a, 28b can have any configurations that are suitable to generate transmit signals to be transmitted by the transmit antennas 22a, 22b. Transmit circuits for generating transmit signals in the RF or microwave frequency range are well known in the art. In one embodiment, the transmit circuits 28a, 28b can include, for example, a connection to a power source, a frequency generator, and optionally filters, amplifiers or any other suitable elements for a circuit generating RF or microwave frequency electromagnetic signals. In an embodiment, the signals generated by the transmit circuits 28a, 28b can have at least two discrete frequencies (i.e. a plurality of discrete frequencies), each of which is in the range from about 10 kHz to about 100 GHz. In another embodiment, each of the at least two discrete frequencies can be in a range from about 300 MHz to about 6000 MHz. In an embodiment, the transmit circuits 28a, 28b can be configured to sweep through a range of frequencies that are within the range of about 10 kHz to about 100 GHz, or in another embodiment a range of about 300 MHz to about 6000 MHz. In an embodiment, the transmit circuits 28a, 28b can be configured to produce complex transmit signals, the complex signals including a plurality of signal components, each of the signal components having a different frequency. The complex signal can be generated by blending or multiplexing multiple signals together followed by transmitting the complex signals whereby the plurality of frequencies are transmitted at the same time.

The receive antennas 26a, 26b are positioned, arranged, and configured to detect the electromagnetic response signals 24a, 24b that result from the transmission of the transmit signals 20a, 20b by the transmit antennas 22a, 22b into the target 12 and impinging on the analyte 14. Each one of the receive antennas 26a, 26b can be an electrode or any other suitable receiver of electromagnetic signals in the radio frequency (RF) or microwave range. In an embodiment, the receive antennas 26aa, 26b are configured to detect electromagnetic signals having at least two frequencies, each of which is in the range from about 10 kHz to about 100 GHz, or in another embodiment a range from about 300 MHz to about 6000 MHz. The receive antennas 26a, 26b can have any arrangement and orientation relative to the target 12 that is sufficient to allow detection of the response signals 24a, 24b to allow the analyte sensing to take place. In one non-limiting embodiment, the receive antennas 26a, 26b can be arranged to face in a direction that is substantially toward the target 12.

The receive circuits 30a, 30b are electrically connectable to the respective receive antennas 26a, 26b and convey the received responses from the receive antennas 26a, 26b to the controllers 32a, 32b. The receive circuits 30a, 30b can have any configuration that is suitable for interfacing with the receive antennas 26a, 26b to convert the electromagnetic energy detected by the receive antennas 26a, 26b into signals reflective of the response signals 24a, 24b. The construction of receive circuits are well known in the art. The receive circuits 26a, 26b can be configured to condition the signals prior to providing the signals to the controllers 32a, 32b, for example through amplifying the signal(s), filtering the signal(s), or the like. Accordingly, the receive circuits 30a, 30b may include filters, amplifiers, or any other suitable components for conditioning the signals provided to the controllers 32a, 32b. In an embodiment, at least one of the receive circuits 30a, 30b or the controllers 32a, 32b can be configured to decompose or demultiplex a complex signal, detected by the receive antennas 26a, 26b, including a plurality of signal components each at different frequencies into each of the constituent signal components. In an embodiment, decomposing the complex signals can include applying a Fourier transform to the detected complex signals. However, decomposing or demultiplexing a received complex signal is optional. Instead, in an embodiment, the complex signals detected by the receive antennas 26a, 26b can be analyzed as a whole (i.e. without demultiplexing the complex signal) to detect the analyte(s) as long as the detected signal provides enough information to make the analyte detection.

The controllers 32a, 32b control the operation of the sensor. The controllers 32a, 32b, for example, can direct the transmit circuits 28a, 28b to generate transmit signals to be transmitted by the transmit antennas 22a, 22b. The controllers 32a, 32b further receive signals from the receive circuits 30a, 30b. The controllers 32a, 32b can optionally process the signals from the receive circuits 30a, 30b to detect the analyte(s) 14 in the target 12. In one embodiment, the controllers 32a, 32b may optionally be in communication with at least one external device 36 such as a user device and/or a remote server 38, for example through one or more wireless connections such as Bluetooth, wireless data connections such a 4G, 5G, LTE or the like, or Wi-Fi. If provided, the external device 36 and/or remote server 38 may process (or further process) the signals that the controllers 32a, 32b receive from the receive circuits 30a, 30b, for example to detect the analyte(s) 14. If provided, the external device 36 may be used to provide communication between the sensor and the remote server 38, for example using a wired data connection or via a wireless data connection or Wi-Fi of the external device 36 to provide the connection to the remote server 38.

With continued reference to FIG. 1, the housing 34 defines an interior space 40. Components of the sensor may be attached to and/or disposed within the housing 34. For example, the transmit antennas 22a, 22b and the receive antennas 26a, 26b are attached to the housing 34. In some embodiments, the antennas 22a, 22b, 26a, 26b may be entirely or partially within the interior space 40 of the housing 34. In some embodiments, the antennas 22a, 22b, 26a, 26b may be attached to the housing 34 but at least partially or fully located outside the interior space 40. In some embodiments, the transmit circuits 28a, 28b, the receive circuits 30a, 30b and the controllers 32a, 32b are attached to the housing 34 and disposed entirely within the sensor housing 34.

The receive antennas 26a, 26b are decoupled or detuned with respect to the transmit antennas 22a, 22b such that electromagnetic coupling between the transmit antennas 22a, 22b and the receive antennas 26a, 26b are reduced. The decoupling of the transmit antennas 22a, 22b and the receive antennas 26a, 26b increases the portion of the signals 24a, 24b detected by the receive antennas 22a, 22b, and minimizes direct receipt of the transmitted signals 20a, 20b by the receive antennas 26a, 26b. The decoupling of the transmit antennas 22a, 22b and the receive antennas 26a, 26b results in transmission from the transmit antennas 22a, 22b to the receive antennas 26a, 26b having a reduced forward gain (S21) and an increased reflection at output (S22) compared to antenna systems having coupled transmit and receive antennas.

In an embodiment, coupling between the transmit antennas 22a, 22b and the receive antennas 26a, 26b is 95% or less. In another embodiment, coupling between the transmit antennas 22a, 22b and the receive antennas 26a, 26b is 90% or less. In another embodiment, coupling between the transmit antennas 22a, 22b and the receive antennas 26a, 26b is 85% or less. In another embodiment, coupling between the transmit antennas 22a, 22b and the receive antennas 26a, 26b is 75% or less.

Any technique for reducing coupling between the transmit antennas 22a, 22b and the receive antennas 26a, 26b can be used. For example, the decoupling between the transmit antennas 22a, 22b and the receive antennas 26a, 26b can be achieved by one or more intentionally fabricated configurations and/or arrangements between the transmit antennas 22a, 22b and the receive antennas 26a, 26b that is sufficient to decouple the transmit antennas 22a, 22b and the receive antennas 26a, 26b from one another. Examples of decoupled transmit and receive antennas that can be used are disclosed in U.S. Pat. Nos. 11,063,373, 11,058,331 and 11,033,208, each of which is incorporated by reference in its entirety.

In one embodiment, the transmit signals 20a, 20b that are transmitted by the transmit antennas 22a, 22b can have at least two different frequencies, for example upwards of 7 to 12 different and discrete frequencies. In another embodiment, the transmit signals 20a, 20b can be a series of discrete, separate signals with each separate signal having a single frequency or multiple different frequencies. In one embodiment, each of the transmit signals 20a, 20b can be transmitted over a transmit time that is less than, equal to, or greater than about 300 ms. In another embodiment, the transmit time can be less than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signals can be transmitted multiple times, and then the transmit time can be averaged. In another embodiment, each one of the transmit signals can be transmitted with a duty cycle that is less than or equal to about 50%.

In one example operation of the sensor in FIG. 1, the sensor assembly 16 and the sensor assembly 18 can be controlled to alternate with one another in emitting the transmit signals 20a, 20b into the target 12 while implementing a frequency sweep over a range of frequencies. For example, in one frequency sweep, the sensor assembly 16 can emit a first one of the transmit signals at a frequency of about 300 MHz, followed by the sensor assembly 18 emitting a transmit signal at a frequency of about 310 MHz, followed by the sensor assembly 16 emitting a transmit signal at a frequency of about 320 MHz, followed by the sensor assembly 18 emitting a transmit signal at a frequency of about 330 MHz, etc. up to an ending frequency of the desired frequency range.

In another example operation of the sensor in FIG. 1, the sensor assembly 16 and the sensor assembly 18 can be controlled so that the sensor assembly 16 and the sensor assembly 18 simultaneously or substantially simultaneously emit the transmit signals. In this embodiment, the frequency of the signal emitted by the sensor assembly 16 can be the same as the frequency of the signal emitted by the sensor assembly 18, or the frequency of the signal emitted by the sensor assembly 16 can be different from the frequency of the signal emitted by the sensor assembly 18.

In another example operation of the sensor in FIG. 1, the sensor assembly 16 and the sensor assembly 18 can be controlled so that the sensor assembly 16 and the sensor assembly 18 detect a common analyte in the target. For example, each sensor assembly 16, 18 can detect an analyte such as glucose in the target 12. In this embodiment, the analyte reading from one of the sensor assemblies 16, 18 can be compared to the analyte reading from the other sensor assembly to validate the analyte reading. Alternatively, the analyte readings from the sensor assemblies 16, 18 can be averaged together in order to obtain an average analyte reading.

In another example, one of the sensor assemblies 16, 18 can detect an analyte in a first part of the target, such as detecting an analyte in blood, while the other sensor assembly 16, 18 can detect an analyte in a second part of the target, for example detecting the same or different analyte in interstitial fluid of the target. Alternatively, one sensor assembly 16, 18 can be used to detect an analyte from one depth of the target, such as the human body, while the other sensor assembly 16, 18 can be used to detect an analyte from a different depth of the human body.

In another example operation of the sensor in FIG. 1, the sensor assembly 16 and the sensor assembly 18 can be controlled so that the sensor assembly 16 detects one analyte, such as glucose, while the sensor assembly 18 detects a different analyte, such as alcohol, in the target.

In an embodiment, the sensor assemblies 16, 18 can be spaced apart from each other by a minimum distance. For example, the minimum spacing distance can be at least 2×the length of one of the antennas 22a, 22b, 26a, 26b. In another embodiment, the minimum spacing distance can be at least 5×the length of one of the antennas 22a, 22b, 26a, 26b.

Other uses of the sensor assemblies 16, 18 are possible, including any combinations of the example operations described herein.

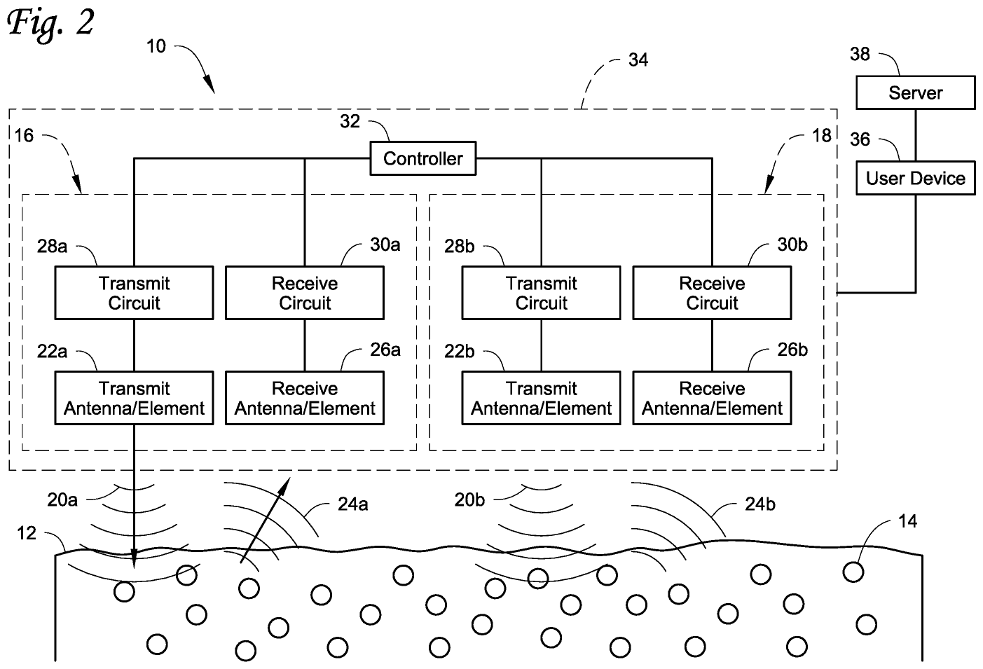

FIG. 2 illustrates another embodiment of a non-invasive analyte sensor system. In FIG. 2, elements that are the same as or similar to elements in the sensor system in FIG. 1 are referenced using the same reference numerals. In this embodiment, the non-invasive analyte sensor 10 is substantially identical to the sensor 10 in FIG. 1. However, instead of using separate controllers 32a, 32b as in FIG. 1, the sensor assemblies 16, 18 in FIG. 2 share a common controller 32. The sensor system in FIG. 2 is otherwise illustrated as being the same as the sensor in FIG. 1.

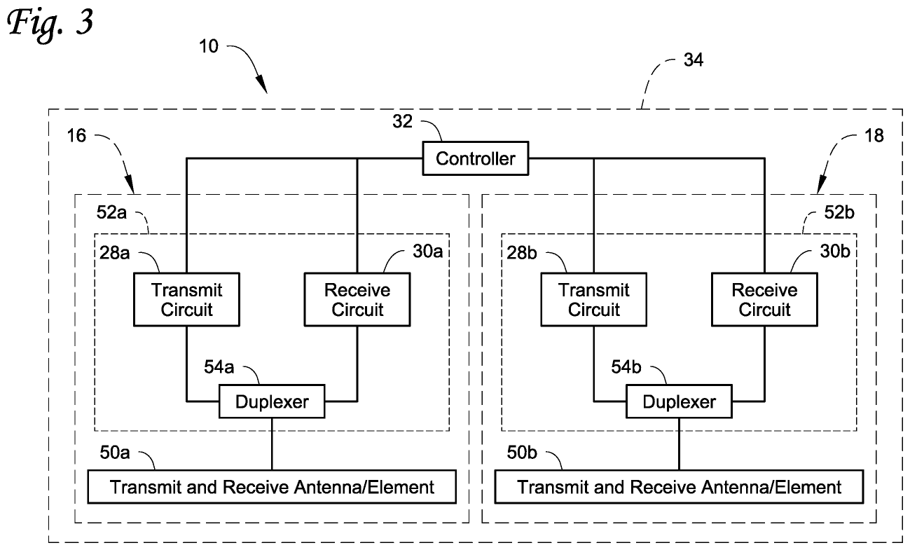

FIG. 3 illustrates another embodiment of a non-invasive analyte sensor system. In FIG. 3, elements that are the same as or similar to elements in the sensor systems in FIGS. 1 and 2 are referenced using the same reference numerals. In the sensor system in FIG. 3, each of the sensor assemblies 16, 18 includes a single antenna/element 50a, 50b that both transmits signals and receives signals. For each assembly 16, 18, a full duplex communication system 52a, 52b is connected to the single antenna 50a, 50b. Each of the sensor assemblies 16, 18, as well as the analyte sensor 10 itself, may also be described as being full duplex or described as being configured for bi-directional communication using the single antennas 50a, 50b. Each one of the full duplex communication systems 52a, 52b is a system that permits the respective single antenna 50a, 50b to both transmit and receive the radio frequency signals. Full duplex communication is defined as simultaneous or near simultaneous data transmission and detection by the antennas 50a, 50b (i.e. signals are simultaneously or near simultaneously transmitted and detected). The antennas 50a, 50b can have a construction similar to the antennas 22a, 26a, 22b, 26b in FIG. 1.

The full duplex communication system 52a, 52b can have any configuration that permits the antennas 50a, 50b to perform simultaneous or near simultaneous signal transmission and detection. For example, the full duplex communication systems 52a, 52b can have a configuration as described in U.S. Patent Application Publication No. 2020/0099504 the entire contents of which are incorporated herein by reference. The full duplex communication system 52a, 52b can also have a configuration as described in “Single Antenna Full Duplex Communications Using A Common Carrier” by Michael Knox, the entire contents of which are incorporated herein by reference.

In the example depicted in FIG. 3, the full duplex communication systems 52a, 52b are shown as including the transmit circuits 28a, 28b, the receive circuits 30a, 30b, and duplexers 54a, 54b. The duplexers 54a, 54b can be any type of device or combination of devices that allows the transmit circuits 28a, 28b and the receive circuits 30a, 30b to share the respective antennas 50a, 50b and perform transmission of the signals and detection of the return signals using the antennas 50a, 50b. Many example of duplexers 54a, 54b that can be used exist. For example, the duplexer can be a switch, a circulator, a hybrid, or an orthomode transducer.

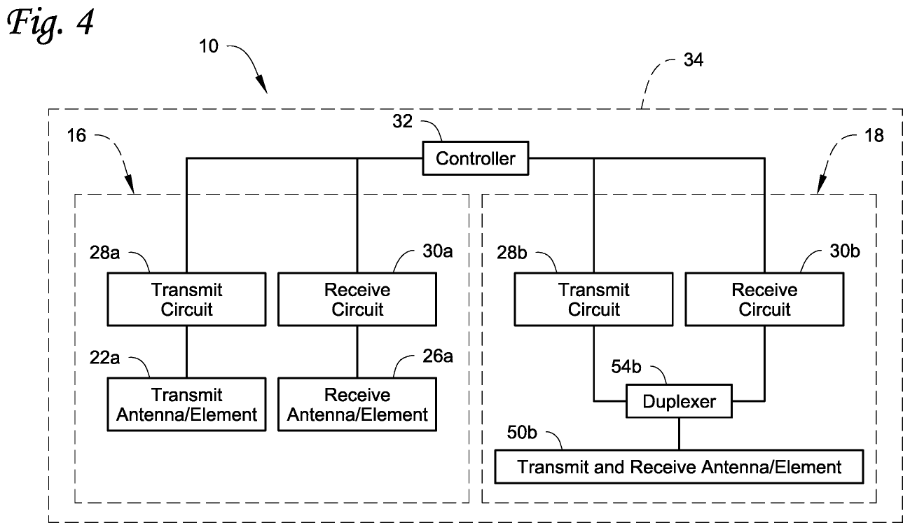

FIG. 4 illustrates another embodiment of a non-invasive analyte sensor system. In FIG. 4, elements that are the same as or similar to elements in the sensor system in FIGS. 1-3 are referenced using the same reference numerals. In this embodiment, the non-invasive analyte sensor 10 combines features of FIGS. 1-2 and FIG. 3. For example, the sensor assembly 16 can have a construction like in FIGS. 1-2, while the sensor assembly 18 can have a construction like in FIG. 3 with the single antenna 50b. Alternatively, the sensor assembly 16 can have a construction like in FIG. 3 with the single antenna 50a, while the sensor assembly 18 can have a construction like in FIGS. 1-2.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A non-invasive analyte sensor, comprising:
first and second non-invasive analyte sensor assemblies, each of the first and second non-invasive analyte sensor assemblies is configured to emit transmit signals that are in a radio or microwave frequency range of the electromagnetic spectrum into a target and configured to detect responses resulting from emission of the transmit signals into the target; and
each of the first and second non-invasive analyte sensor assemblies are disposed at a same side of the target.

2. The non-invasive analyte sensor of claim 1, wherein each of the first and second non-invasive analyte sensor assemblies comprises:
at least one transmit antenna and at least one receive antenna that are decoupled from one another, the at least one transmit antenna is positioned and arranged to transmit the transmit signals into the target, and the at least one receive antenna is positioned and arranged to detect the responses;
a transmit circuit that is electrically connectable to the at least one transmit antenna, the transmit circuit is configured to generate the transmit signals; and
a receive circuit that is electrically connectable to the at least one receive antenna, the receive circuit is configured to receive the responses detected by the at least one receive antenna

3. The non-invasive analyte sensor of claim 1, wherein the first non-invasive analyte sensor assembly and the second non-invasive analyte sensor assembly are disposed in a sensor housing.

4. The non-invasive analyte sensor of claim 3, wherein the first non-invasive analyte sensor assembly and the second non-invasive analyte sensor assembly are disposed side-by-side in the sensor housing.

5. The non-invasive analyte sensor of claim 1, wherein the first non-invasive analyte sensor assembly is controlled by a first controller, and the second non-invasive analyte sensor assembly is controlled by a second controller.

6. The non-invasive analyte sensor of claim 1, wherein the first non-invasive analyte sensor assembly and the second non-invasive analyte sensor assembly are controlled by a common controller.

7. The non-invasive analyte sensor of claim 1, wherein each of the transmit signals emitted by the first non-invasive analyte sensor assembly has a frequency that is in a range that is between about 10 kHz to about 100 GHz; and each of the transmit signals emitted by the second non-invasive analyte sensor assembly has a frequency that is in a range that is between about 10 kHz to about 100 GHz.

8. The non-invasive analyte sensor of claim 1, wherein the first non-invasive analyte sensor assembly and the second non-invasive analyte sensor assembly are controlled to alternate with one another in emitting the transmit signals into the target while implementing a frequency sweep over a range of frequencies.

9. The non-invasive analyte sensor of claim 1, wherein the first non-invasive analyte sensor assembly and the second non-invasive analyte sensor assembly are controlled to simultaneously emit the transmit signals.

10. The non-invasive analyte sensor of claim 1, wherein the first non-invasive analyte sensor assembly and the second non-invasive analyte sensor assembly are configured to detect a common analyte in the target.

11. The non-invasive analyte sensor of claim 1, wherein the first non-invasive analyte sensor assembly and the second non-invasive analyte sensor assembly are configured to detect different analytes in the target.

12. A non-invasive analyte sensor, comprising:
a first non-invasive analyte sensor assembly that is configured to emit first transmit signals that are in a radio or microwave frequency range of the electromagnetic spectrum into a first portion of a target and configured to detect first responses resulting from emission of the first transmit signals into the first portion of the target; and
a second non-invasive analyte sensor assembly that is configured to emit second transmit signals that are in a radio or microwave frequency range of the electromagnetic spectrum into a second portion of the target and configured to detect second responses resulting from emission of the second transmit signals into the second portion of the target, the second portion of the target is spaced from the first portion of the target.

13. The non-invasive analyte sensor of claim 12, wherein the first non-invasive analyte sensor assembly comprises:
at least one first transmit antenna and at least one first receive antenna that are decoupled from one another, the at least one first transmit antenna is positioned and arranged to transmit the first transmit signals into the first portion of the target, and the at least one first receive antenna is positioned and arranged to detect the first responses;
a first transmit circuit that is electrically connectable to the at least one first transmit antenna, the first transmit circuit is configured to generate the first transmit signals; and
a first receive circuit that is electrically connectable to the at least one first receive antenna, the first receive circuit is configured to receive the first responses detected by the at least one first receive antenna;
and the second non-invasive analyte sensor assembly comprises:
at least one second transmit antenna and at least one second receive antenna that are decoupled from one another, the at least one second transmit antenna is positioned and arranged to transmit the second transmit signals into the second portion of the target, and the at least one second receive antenna is positioned and arranged to detect the second responses;
a second transmit circuit that is electrically connectable to the at least one second transmit antenna, the second transmit circuit is configured to generate the second transmit signals; and
a second receive circuit that is electrically connectable to the at least one second receive antenna, the second receive circuit is configured to receive the second responses detected by the at least one second receive antenna.

14. The non-invasive analyte sensor of claim 12, wherein the first non-invasive analyte sensor assembly and the second non-invasive analyte sensor assembly are disposed in a sensor housing.

15. The non-invasive analyte sensor of claim 14, wherein the first non-invasive analyte sensor assembly and the second non-invasive analyte sensor assembly are disposed side-by-side in the sensor housing.

16. The non-invasive analyte sensor of claim 12, wherein the first non-invasive analyte sensor assembly is controlled by a first controller, and the second non-invasive analyte sensor assembly is controlled by a second controller.

17. The non-invasive analyte sensor of claim 12, wherein the first non-invasive analyte sensor assembly and the second non-invasive analyte sensor assembly are controlled by a controller.

18. The non-invasive analyte sensor of claim 12, wherein each of the first transmit signals emitted by the first non-invasive analyte sensor assembly has a frequency that is in a range that is between about 10 kHz to about 100 GHz; and each of the second transmit signals emitted by the second non-invasive analyte sensor assembly has a frequency that is in a range that is between about 10 kHz to about 100 GHz.

19. The non-invasive analyte sensor of claim 12, wherein the first non-invasive analyte sensor assembly and the second non-invasive analyte sensor assembly are controlled to alternate with one another in emitting the transmit signals into the target while implementing a frequency sweep over a range of frequencies.

20. The non-invasive analyte sensor of claim 12, wherein the first non-invasive analyte sensor assembly and the second non-invasive analyte sensor assembly are configured to simultaneously emit the first transmit signals and the second transmit signals.

21. The non-invasive analyte sensor of claim 12, wherein the first non-invasive analyte sensor assembly and the second non-invasive analyte sensor assembly are configured to detect a common analyte in the target.

22. The non-invasive analyte sensor of claim 12, wherein the first non-invasive analyte sensor assembly and the second non-invasive analyte sensor assembly are configured to detect different analytes in the target.
